(19) United States
(12) Patent Application Publication
(10) Pub. No.: US 2023/0008809 A1
(43) Pub. Date: Jan. 12, 2023

(54) SYSTEMS AND METHODS FOR ENHANCING INFECTION DETECTION AND MONITORING THROUGH DECOMPOSED PHYSIOLOGICAL DATA

(71) Applicant: THE REGENTS OF THE UNIVERSITY OF MICHIGAN, Ann Arbor, MI (US)

(72) Inventors: Daniel Forger, Ann Arbor, MI (US); Jonathan Tyler, Whitmore Lake, MI (US); Caleb Mayer, Ann Arbor, MI (US); Srijan Sen, Ann Arbor, MI (US); Yu Fang, Ann Arbor, MI (US); Christopher Flora, Temperance, MI (US); Sung Won Choi, Ann Arbor, MI (US); Muneesh Tewari, Ann Arbor, MI (US)

(21) Appl. No.: 17/859,647

(22) Filed: Jul. 7, 2022

Related U.S. Application Data
(60) Provisional application No. 63/219,139, filed on Jul. 7, 2021.

(51) Int. Cl. A61B 5/22

(52) U.S. Cl. CPC A61B 5/222; A63B 2230/06

(57) ABSTRACT

Systems and methods for enhancing infection detection and monitoring through decomposed physiological data are disclosed. An example method includes receiving, from a wearable device of a user, physiological data of the user and decomposing the physiological data, by applying a heart rate algorithm, to generate one or more physiological parameters. The example method further includes analyzing, by applying the heart rate algorithm, the one or more physiological parameters to output a period classification, and determining whether or not the period classification is indicative of an infection. The example method further includes, responsive to determining that the period classification is indicative of the infection, displaying, in a user interface, a warning to the user that indicates the infection, and receiving, from the wearable device of the user, additional physiological data of the user to monitor the infection.

### FIELD OF THE DISCLOSURE

The present disclosure relates generally to techniques for detecting infections and, more particularly, to techniques for enhancing infection detection and monitoring through decomposed physiological data.

### BACKGROUND

The background description provided herein is for the purpose of generally presenting the context of the disclosure. Work of the presently named inventor, to the extent it is described in this background section, as well as aspects of the description that may not otherwise qualify as prior art at the time of filing, are neither expressly nor impliedly admitted as prior art against the present disclosure.

Infections are generally harmful to patients and may cause a wide variety of deleterious effects to a patient's well-being. Respiratory infections, in particular, can produce several debilitating symptoms and may cause lasting damage to a patient's health. For example, the global COVID-19 pandemic has infected millions across the planet, and causes a litany of symptoms including fever, shortness of breath, fatigue, congestion, nausea, and even loss of taste or smell. Unfortunately, these infections can be highly contagious, and are potentially life-threatening to people of all ages if left undetected, untreated, and/or unmonitored.

Conventionally, there are numerous techniques for detecting and monitoring infections. These techniques range from formal hospital settings that monitor patient vital signs using advanced medical technology to informal patient self-assessments. Problematically, each of these conventional techniques suffer from several drawbacks. Formal hospital settings and tests conducted therein are incredibly expensive and time-intensive, and the medical equipment itself generally lacks predictive power, leaving such analysis to the informed guesses of experienced physicians examining the results. By contrast, patient self-assessments generally provide underdeveloped views of a patient's health, which when combined with an average patient's lack of understanding of medical principles, can lead patients to underappreciate/neglect certain biological markers of infection.

More recently, with the advances of physiological sensors integrated into consumer wearable devices, patients and/or medical providers may utilize the data from these wearables to assess a patient's general health. Most wearables include sensors capable of detecting a patient's heart rate and activity level (most commonly in the form of steps taken). This data is easily accessible, and provides a relatively complete record of a patient's heart rate and activity levels across days/weeks/months etc. As a result, consumer wearable device data has been integrated into several conventional techniques to provide useful insights related to a patient's cardiac health, daily activity levels, and other broad health indicators. However, this integration has not enabled the conventional techniques to evaluate a patient's risk of developing and/or recovering from an infection with any greater effectiveness. Moreover, utilizing this data as part of the conventional techniques (e.g., visiting a doctor) subjects the patient to many of the shortcomings associated with the conventional techniques (e.g., high prices, long wait times, general inconvenience) without use of the wearable consumer device data.

Therefore, there is a need for techniques capable of accurately and efficiently detecting the onset of an infection and monitoring the infection within a patient utilizing the physiological data collected by a consumer wearable device.

### SUMMARY OF THE INVENTION

According to an aspect of the present disclosure, a method for enhancing detection and monitoring through decomposed physiological data includes receiving, from a wearable device of a user, a first set of physiological data of the user, wherein the first set of physiological data of the user comprises at least heart rate data of the user during a first period. The method further includes decomposing, by one or more processors applying a heart rate algorithm, the first set of physiological data of the user to generate one or more physiological parameters, each of the one or more physiological parameters corresponding to one or more respective physiological systems of the user; analyzing, by the one or more processors applying the heart rate algorithm, the one or more physiological parameters to output a period classification; determining, by the one or more processors, whether or not the period classification is indicative of an infection; responsive to determining that the period classification is indicative of the infection, displaying, in a user interface, a warning to the user that indicates the infection; and receiving, from the wearable device of the user, a second set of physiological data of the user to monitor the infection, wherein the second set of physiological data of the user includes at least the heart rate data of the user during a second period.

In some arrangements, the one or more physiological parameters comprises at least (i) an average heart rate of the user and one or more of: (ii) a circadian variation amplitude, (iii) a circadian variation phase, (iv) an activity parameter, (v) an autocorrelated noise parameter, or (vi) an uncorrelated noise parameter.

In certain arrangements, the first set of physiological data of the user comprises the heart rate of the user, an active step number of the user, and a temperature of the user.

In some arrangements, the method further comprises responsive to determining that the period classification is indicative of the infection, determining, by the one or more processors, a recovery time of the user from the infection based on at least one respective physiological parameter of the one or more physiological parameters. In these arrangements, the one or more physiological parameters comprises a first set of one or more physiological parameters. Additionally, the method further comprises decomposing, by one or more processors applying the heart rate algorithm, the second set of physiological data of the user to generate a second set of one or more physiological parameters, wherein each of the second set of one or more physiological parameters corresponds to the one or more respective physiological systems of the user; and updating, by the one or more processors, the recovery time of the user based on the second set of one or more physiological parameters.

In certain arrangements, decomposing the first set of physiological data of the user further comprises determining, by the one or more processors applying the heart rate algorithm, a set of sleep heart rate data from the first set of physiological data of the user that was captured by the wearable device of the user during a sleep period of the first period; and removing, by the one or more processors, the set of sleep heart rate data from the first set of physiological data of the user.

In some arrangements, the first set of physiological data comprises a plurality of first physiological data points, each respective first physiological data point is binned into one of a plurality of bins, and each respective bin represents a five minute interval of the first period.

In certain arrangements, analyzing the one or more physiological parameters further comprises comparing, by the one or more processors applying the heart rate algorithm, the one or more physiological parameters to a user-specific baseline; determining, by the one or more processors applying the heart rate algorithm, whether or not any deviations of the one or more physiological parameters from the user-specific baseline exceed an infection threshold; and responsive to determining that at least one deviation of a respective physiological parameter from the user-specific baseline exceeds the infection threshold, generating, by the one or more processors, the warning to the user that indicates the infection. In these arrangements, the user-specific baseline includes one or more respective baseline physiological parameters, each of the one or more respective baseline physiological parameters corresponding to a respective physiological parameter of the one or more physiological parameters.

In some arrangements, the one or more respective physiological systems of the user each produce a physiological impact on the heart rate of the user, and the one or more respective physiological systems comprise one or more of: (i) circadian timekeeping, (ii) respiratory function related to increased heart rate from activity of the user, (iii) sleep, (iv) hormones, (v) meal consumption, (vi) caffeine intake, or (vii) posture.

In certain arrangements, the heart rate algorithm is an artificial intelligence (AI) based algorithm, and the method further comprises training, by the one or more processors, the heart rate algorithm with a set of healthy period training data and a set of symptomatic period training data to output respective physiological parameters and respective period classifications. In these arrangements, the heart rate algorithm is a linear support-vector machine (SVM) model.

According to another aspect of the present disclosure, a system for enhancing infection detection and monitoring through decomposed physiological data comprises a user interface, a wearable device of a user, a memory, and a processor interfacing with the wearable device, the memory, and the user interface. The wearable device of the user is configured to capture a first set of physiological data of the user, wherein the first set of physiological data of the user comprises at least heart rate data of the user during a first period. The memory stores a set of computer-readable instructions comprising at least a heart rate algorithm. The processor is configured to execute the set of computer-readable instructions to cause the processor to receive, from the wearable device of the user, the first set of physiological data of the user; decompose, by applying the heart rate algorithm, the first set of physiological data of the user to generate one or more physiological parameters, each of the one or more physiological parameters corresponding to one or more respective physiological systems of the user; analyze, by applying the heart rate algorithm, the one or more physiological parameters to output a period classification; determine whether or not the period classification is indicative of an infection; responsive to determining that the period classification is indicative of the infection, display, in the user interface, a warning to the user that indicates the infection; and receive, from the wearable device of the user, a second set of physiological data of the user to monitor the infection, wherein the second set of physiological data of the user includes at least the heart rate data of the user during a second period.

In certain arrangements, the one or more physiological parameters comprises at least (i) an average heart rate of the user and one or more of: (ii) a circadian variation amplitude, (iii) a circadian variation phase, (iv) an activity parameter, (v) an autocorrelated noise parameter, or (vi) an uncorrelated noise parameter.

In some arrangements, the first set of physiological data of the user comprises the heart rate of the user, an active step number of the user, and a temperature of the user.

In certain arrangements, the set of computer-readable instructions further cause the processor to responsive to determining that the period classification is indicative of the infection, determine a recovery time of the user from the infection based on at least one respective physiological parameter of the one or more physiological parameters.

In some arrangements, the set of computer-readable instructions further cause the processor to determine, by applying the heart rate algorithm, a set of sleep heart rate data from the first set of physiological data of the user that was captured by the wearable device of the user during a sleep period of the first period; and remove the set of sleep heart rate data from the first set of physiological data of the user.

In certain arrangements, the first set of physiological data comprises a plurality of first physiological data points, each respective first physiological data point is binned into one of a plurality of bins, and each respective bin represents a five minute interval of the first period.

In some arrangements, the set of computer-readable instructions further cause the processor to compare the one or more physiological parameters to a user-specific baseline, wherein the user-specific baseline includes one or more respective baseline physiological parameters, each of the one or more respective baseline physiological parameters corresponding to a respective physiological parameter of the one or more physiological parameters; determine whether or not any deviations of the one or more physiological parameters from the user-specific baseline exceed an infection threshold; and responsive to determining that at least one deviation of a respective physiological parameter from the user-specific baseline exceeds the infection threshold, generate the warning to the user that indicates the infection.

According to yet another aspect of the present disclosure, a non-transitory computer-readable storage medium has stored thereon a set of instructions, executable by at least one processor, for enhancing infection detection and monitoring through decomposed physiological data. The instructions comprise instructions for accessing a first set of physiological data of a user, wherein the first set of physiological data of the user comprises at least heart rate data of the user during a first period; instructions for decomposing, by applying a heart rate algorithm, the first set of physiological data of the user to generate one or more physiological parameters, each of the one or more physiological parameters corresponding to one or more respective physiological systems of the user; instructions for analyzing, by applying the heart rate algorithm, the one or more physiological parameters to output a period classification; instructions for determining whether or not the period classification is indicative of an infection; responsive to determining that the period classification is indicative of the infection, instructions for displaying, in a user interface, a warning to the user that indicates the infection; and instructions for accessing a second set of physiological data of the user to monitor the infection, wherein the second set of physiological data of the user includes at least the heart rate data of the user during a second period.

### BRIEF DESCRIPTION OF THE DRAWINGS

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1A illustrates an example system for enhancing infection detection and monitoring through decomposed physiological data, in accordance various aspects disclosed herein.

FIG. 1B illustrates an example parameter fit corresponding to physiological parameters obtained from a wearable device of a user, in accordance with various aspects disclosed herein.

FIG. 1C illustrates an example physiological data collection and parameterization timeline corresponding to the example system of FIG. 1A, and in accordance with various aspects disclosed herein.

FIG. 2A is a graph illustrating an example infection detection utilizing the wearable device of the example system of FIG. 1A, and in accordance with various aspects disclosed herein.

FIG. 2B is a graph illustrating example changes in a user's heart rate, as measured by the wearable device of the example system of FIG. 1A, that occur during and after the onset of an infection, in accordance with various aspects disclosed herein.

FIG. 2C is a graph illustrating example changes in a user's heart rate due to steps, as measured by the wearable device of the example system of FIG. 1A, that occur during and after the onset of an infection, in accordance with various aspects disclosed herein.

FIG. 2D is a graph illustrating example changes in the autocorrelated noise of a user's physiological data, as included in measurements of the wearable device of the example system of FIG. 1A, that occur during and after the onset of an infection, in accordance with various aspects disclosed herein.

FIG. 3A is a graph illustrating an example recovery time estimation, generated by the example system of FIG. 1A, based on a user's maximum increase in heart rate per step during the infection, in accordance with various aspects disclosed herein.

FIG. 3B is a graph illustrating another example recovery time estimation, generated by the example system of FIG. 1A, based on a user's maximum phase uncertainty increase during the infection, in accordance with various aspects disclosed herein.

FIG. 4 illustrates an example method for enhancing infection detection and monitoring through decomposed physiological data, in accordance various aspects disclosed herein.

FIG. 5A illustrates an example user interface as rendered on a display screen of a user device, in accordance with various aspects disclosed herein.

FIG. 5B illustrates another example user interface as rendered on a display screen of a wearable device of the user, in accordance with various aspects disclosed herein.

### DETAILED DESCRIPTION

Generally, wearable consumer devices (also referenced herein as “wearables” and “wearable devices”) have emerged as a key tool in the fight against pandemics and respiratory infections. Wearables provide a convenient, low-cost solution to obtain user-specific physiological data. Moreover, wearables may enable more frequent and more remote (e.g., at-home) monitoring of a user's health status, which can be crucial during periods before and during a user's infection, especially during a pandemic. Using the systems and methods of the present disclosure, these wearables can detect an infection prior to symptom onset and can be used once symptoms develop to monitor and predict recovery times for specific infections, such as COVID-19.

Physiological signals that are measured by wearables, such as but not limited to heart rate, temperature, and steps, reflect the combined action of many physiological and environmental signals on a user's health. For example, circadian timekeeping, the effect of activity of steps on heart rate and temperature, the effect of sleep, cortisol or other hormones, and the physiological effects of meals, drugs including caffeine and posture all play an important role in the resulting physiological signals measured by the wearable devices. The systems and methods of the present disclosure generally decompose the physiological signals measured by the wearable to determine signals corresponding to different physiological systems, and use these separate signals both to detect and monitor the course of infection of a user. By decomposing these signals, the systems and methods of the present disclosure provide increased predictive power for detecting and tracking infections compared to conventional techniques. Moreover, these decomposed signals can be used in home or clinical settings, along with other markers, to identify suitable treatments or triage medical resources in a flexible manner that conventional techniques are incapable of providing.

When the physiological signals are decomposed, they may be used in multiple ways to detect and/or track a user's infection. For example, residuals may be analyzed to detect anomalies, and changes in parameters fitting the signals from a particular physiological system may be similarly analyzed. Additionally, multiple physiological signals, parameters, and/or residuals may be used together in a combined approach, such as a machine learning (ML) model, to detect and/or track the user's infection. To illustrate, the extent (e.g., severity, recovery period, etc.) of a user's respiratory infection may be measured/predicted based on an increased heart rate due to activity. As a result, the systems and methods of the present disclosure improve over conventional techniques by accurately classifying symptomatic versus healthy periods of a user with an area under the curve (AUC) of approximately 0.92.

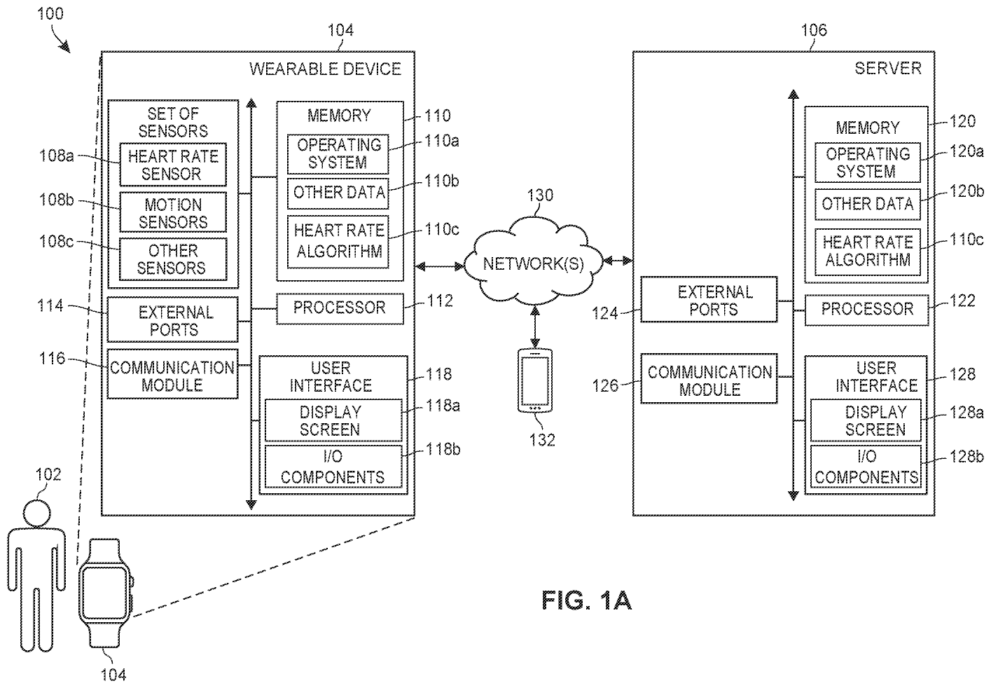

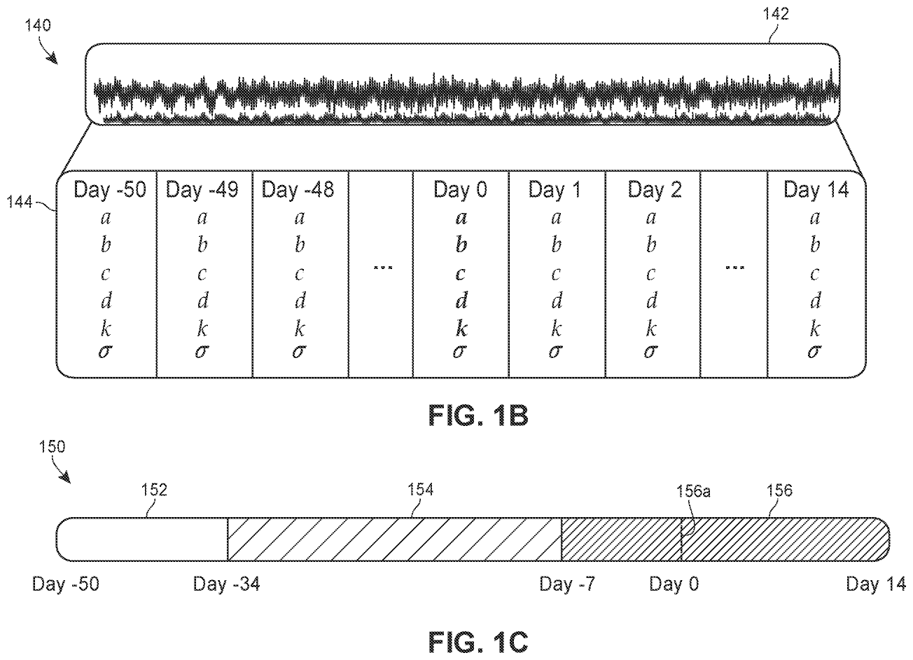

FIG. 1A illustrates an example system 100 for enhancing infection detection and monitoring through decomposed physiological data. It should be appreciated that the example system 100 is merely an example and that alternative or additional components are envisioned.

As illustrated in FIG. 1A, the example system 100 features a user 102 who may wear a wearable device 104 and a server 106. Generally, the wearable device 104 may be any device that is wearable by a user that is configured to monitor and collect physiological data corresponding to the user. For example, the wearable device 104 may be a smartwatch/bracelet (e.g., FITBIT, APPLE Watch, WHOOP Strap, etc.), smart ring, knee bands, arm bands, sensor patches, clothing-based monitors, chest straps, etc. The system 100 may also include other wearable devices which may be worn by the user 102, such as a smart ring in addition to a smartwatch (e.g., wearable device 104).

The wearable device 104 may include a set of sensors 108 that are each configured to detect and/or collect physiological data of the user 102. The set of sensors 108 may include a heart rate sensor 108a (e.g., an optical heart rate sensor) that is configured to detect and collect heart rate data of the user 102 and motion sensor(s) 108b configured to detect and collect motion data corresponding to the user 102. The motion sensor(s) 108b may include, for example, an accelerometer, a gyroscope, an altimeter, a global positioning system (GPS), a magnetometer, an electrodermal activity (EDA) sensor, and/or any other suitable sensor or combinations thereof. The set of sensors 108 may also include other sensors 108c, such as a skin temperature sensor, an ultraviolet (UV) sensor, a gesture sensor, an electrocardiogram (ECG) sensor, a proximity sensor, a bioimpedance sensor, a pulse oximeter (Sp02) sensor, an ambient light sensor, and/or any other suitable sensors or combinations thereof.

The wearable device 104 may include a memory 110 as well as a processor 112. The memory 110 may store an operating system 110a capable of facilitating the functionalities as discussed herein, as well as other data 110b and a heart rate algorithm 110c. Generally, the processor 112 may interface with the memory 110 to access/execute the operating system 110a, the other data 110b, and the heart rate algorithm 110c. The other data 110b may include a set of applications configured to facilitate the functionalities as discussed herein, and/or may include other relevant data, such as display formatting data, etc. For example, the processor 112 may access the operating system 110a in order to execute applications included as part of the other data 110b, such as a fitness application (not shown) configured to facilitate functionalities associated with enhancing infection detection and monitoring through decomposed physiological data, as discussed herein. It should be appreciated that one or more other applications are envisioned.

The heart rate algorithm 110c may be configured to output physiological parameters and/or period classifications that generally correspond to healthy and/or symptomatic periods of a user 102 wearing the wearable device 104 relative to a particular infection. As part of the analysis performed by the heart rate algorithm 110c, the algorithm 110c may generate a user-specific baseline using physiological data collected by the set of sensors 108, against which, subsequent physiological data may be compared. Further, the physiological data collected by the set of sensors 108 may be binned (e.g., in memory 110) and/or averaged into periodic categories (e.g., minutes, hours, days) that the heart rate algorithm 110c may use to evaluate the health of the user 102 on a continuing basis. The heart rate algorithm 110c may periodically access/request/retrieve this physiological data collected by the set of sensors 108 and analyze the physiological data to output physiological parameters and/or a period classification indicating that the user 102 is either healthy (e.g., no observed or predicted symptoms of an infection/illness) or symptomatic (e.g., one or more observed or predicted symptoms of an infection/illness).

For example, if a user 102 is healthy and shows no physiological symptoms associated with an infection (e.g., elevated heart rate), then the heart rate algorithm 110c may generate a period classification indicating that the user 102 is healthy. By contrast, if the user 102 is experiencing physiological symptoms associated with an infection, then the heart rate algorithm 110c may generate a period classification indicating that the user 102 is symptomatic. In certain cases, a user 102 may not experience many or any prominent physiological symptoms associated with an infection, but the heart rate algorithm 110c may analyze the physiological data collected by the set of sensors 108 and conclude that a symptomatic period classification applies to the physiological data of the user 102.

The heart rate algorithm 110c may output the physiological parameters and/or the period classification for display and/or further analysis by the wearable device 104. The wearable device 104 may thereby receive and process user-specific physiological data to locally determine physiological parameters and/or period classifications that may be saved locally in the memory 110 and/or used as part of an application or platform.

Generally, the heart rate algorithm 110c may be an artificial intelligence (AI) based model trained with at least one AI algorithm. Training of the heart rate algorithm 110c involves training data analysis (e.g., healthy period training data and symptomatic period training data) to configure weights of the heart rate algorithm 110c used to predict and/or classify future received physiological data. For example, in various aspects herein, generation of the heart rate algorithm 110c involves training the heart rate algorithm 110c with a plurality of training data representing healthy periods and symptomatic periods of respective users. The training may be performed locally by a user device (e.g., wearable device 104, user device 132); however, in some aspects, one or more processors of a server or a cloud-based computing platform (e.g., server 106) may receive the plurality of training data representing the healthy periods and symptomatic periods of respective users via a computer network (e.g., network 130). In such aspects, the server and/or the cloud-based computing platform may train the heart rate algorithm 110c with the plurality of training data.

As previously mentioned, an AI based model, as described herein (e.g. heart rate algorithm 110c), may be trained using a supervised machine learning program or algorithm, such as support vector machine (SVM). Generally, machine learning may involve identifying and recognizing patterns in existing data (such as generating physiological parameters and/or period classifications corresponding to physiological data received from a wearable device of a user) in order to facilitate making predictions or identification for subsequent data (such as using the algorithm on new physiological data in order to determine or generate physiological parameters and a period classification corresponding to the new physiological data received from the wearable device of the user). Machine learning model(s), such as the AI based model described herein for some aspects, may be created and trained based upon example data (e.g., “training data” and related user-specific data) inputs or data (which may be termed “features” and “labels”) in order to make valid and reliable predictions for new inputs, such as testing level or production level data or inputs.

In supervised machine learning, a machine learning program operating on a server, computing device, or otherwise processor(s), may be provided with example inputs (e.g., “features”) and their associated, or observed, outputs (e.g., “labels”) in order for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based on the discovered rules, relationships, or model, an expected output.

However, while described herein as being trained using a supervised learning technique (e.g., SVM), in certain aspects, the AI based model may be trained using multiple supervised machine learning techniques, and may additionally or alternatively be trained using one or more unsupervised machine learning techniques. In unsupervised machine learning, the server, computing device, or otherwise processor(s), may be required to find its own structure in unlabeled example inputs, where, for example multiple training iterations are executed by the server, computing device, or otherwise processor(s) to train multiple generations of model until a satisfactory model, e.g., a model that provides sufficient prediction accuracy when given test level or production level data or inputs, is generated.

For example, in certain aspects, the AI based model may employ a SVM algorithm, which may be a program that learns using two or more features or feature datasets (e.g., user-specific physiological data). The machine learning programs or algorithms may also include natural language processing, semantic analysis, neural networks, automatic reasoning, decision tree analysis, random forest analysis, K-Nearest neighbor analysis, naïve Bayes analysis, clustering, reinforcement learning, and/or other machine learning algorithms and/or techniques. In some aspects, the artificial intelligence and/or machine learning based algorithms may be included as a library or package executed on the server 106. For example, libraries may include the TENSORFLOW based library, the PYTORCH library, the SCIKIT-LEARN Python library, and/or a MATLAB library.

Regardless, training the AI based model (e.g., heart rate algorithm 110c) may also comprise retraining, relearning, or otherwise updating models with new, or different, information, which may include information received, ingested, generated, or otherwise used over time. Moreover, in various aspects, the AI based model may be trained, by one or more processors (e.g., processor(s) 122 of server 106 and/or processor(s) of a computer user device, such as the wearable device 104 and/or the user device 132) with the plurality of training data of healthy periods and symptomatic periods (e.g., physiological data representing each of the healthy periods and symptomatic periods) of respective individuals. In these aspects, the AI based model may additionally be configured to generate/output respective physiological parameters and respective period classifications corresponding to each of the healthy period training data and the symptomatic period training data of each respective individual included as part of the plurality of training data.

In any event, the memory 110 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others.

The wearable device 104 may further include a communication module 116 configured to communicate data via one or more networks 130. According to some aspects, the communication module 116 may include one or more transceivers (e.g., WWAN, WLAN, and/or WPAN transceivers) functioning in accordance with IEEE standards, 3GPP standards, or other standards, and configured to receive and transmit data via one or more external ports 114. For example, the communication module 116 may communicate with the server 106 via the network(s) 130.

The wearable device 104 may further include a user interface 118 configured to present information to the user 102 and/or receive inputs from the user 102. As shown in FIG. 1A, the user interface 118 may include a display screen 118a and I/O components 118b (e.g., ports, capacitive or resistive touch sensitive input panels, keys, buttons, lights, LEDs). According to some aspects, the user 102 may access the wearable device 104 via the user interface 118 to review warnings displayed as a result of outputs from the heart rate algorithm 110c, physiological data collected by the set of sensors 108, make various selections, and/or otherwise interact with the wearable device 104.

In some aspects, the wearable device 104 may perform the functionalities as discussed herein as part of a “cloud” network or may otherwise communicate with other hardware or software components within the cloud to send, retrieve, or otherwise analyze data.

As illustrated in FIG. 1A, the wearable device 104 may communicate and interface with the server 106 via the network(s) 130. The server 106 may be associated with, for example, an entity that owns, operates, and/or manages a fitness application or platform and/or a medical services provider that maintains medical records for the user 102. In particular, the server 106 may include or support a web server configured to host a website that enables users to operate the fitness application or platform. Further, the server 106 may support a software application executable by the wearable device 104 (i.e., the wearable device 104 may interface with the server 106 in executing the software application). In certain aspects, the network(s) 130 may support any type of data communication via any standard or technology (e.g., GSM, CDMA, TDMA, WCDMA, LTE, EDGE, OFDM, GPRS, EV-DO, UWB, Internet, IEEE 802 including Ethernet, WiMAX, Wi-Fi, Bluetooth, and others).

The server 106 may include a memory 120 as well as a processor 122. The memory 120 may store an operating system 120a capable of facilitating the functionalities as discussed herein as well as other data 120b and the heart rate algorithm 110c. Generally, the processor 122 may interface with the memory 120 to access/execute the operating system 120a, the other data 120b, and the heart rate algorithm 110c. The other data 120b may include data received from the wearable device 104, a set of applications configured to facilitate the functionalities as discussed herein, and/or may include other relevant data, such as display formatting data, etc. For example, the processor 122 may access the operating system 120a in order to execute applications included as part of the other data 120b, such as a fitness application (not shown) configured to facilitate functionalities associated with enhancing infection detection and monitoring through decomposed physiological data, as discussed herein. It should be appreciated that one or more other applications are envisioned.

The memory 120 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. Further, the server 106 may be configured to interface with or support an external memory or storage (not shown) capable of storing various data, such as in one or more databases or other forms of storage. According to certain aspects, the external storage may store data or information associated with user 102 medical records, physiological data, and/or other suitable data corresponding to the systems and methods described herein. For example, the external storage may store heart rate data of the user 102.

The server 106 may further include a communication module 126 configured to communicate data via the network(s) 130. According to some aspects, the communication module 126 may include one or more transceivers (e.g., WWAN, WLAN, and/or WPAN transceivers) functioning in accordance with IEEE standards, 3GPP standards, or other standards, and configured to receive and transmit data via one or more external ports 124.

The server 106 may further include a user interface 128 configured to present information to a user and/or receive inputs from the user. As shown in FIG. 1A, the user interface 128 may include a display screen 128a and I/O components 128b (e.g., ports, capacitive or resistive touch sensitive input panels, keys, buttons, lights, LEDs). According to some aspects, the user 102 may access the server 106 via the user interface 128 to review information (e.g., warnings generated based on period classifications output from the heart rate algorithm 110c, physiological data collected the set of sensors 108), make selections, and/or perform other functions.

In some aspects, the server 106 may perform the functionalities as discussed herein as part of a “cloud” network or may otherwise communicate with other hardware or software components within the cloud to send, retrieve, or otherwise analyze data. Moreover, although depicted as a single server 106 in FIG. 1A, it should be appreciated that the server 106 may be in the form of a distributed cluster of computers, servers, machines, or the like. In this implementation, the entity may utilize the distributed server(s) 106 as part of an on-demand cloud computing platform. Accordingly, when the wearable device 104 interfaces with the server 106, the wearable device 104 may actually interface with one or more of a number of distributed computers, servers, machines, or the like, to facilitate the described functionalities.

Additionally, the wearable device 104 may connect, through the network(s) 130, to the server 106 and a user device 132. The user device 132 may generally be any type of electronic device such as a mobile device (e.g., a smartphone), desktop computer, notebook computer, tablet, phablet, GPS (Global Positioning System) or GPS-enabled device, smart watch, smart glasses, smart bracelet, wearable electronic, PDA (personal digital assistant), pager, computing device configured for wireless communication, and/or the like. The user device 132 may execute or interface with an application or platform that enables the user 102 to view physiological data, medical records, etc. collected by the wearable device 104 and/or stored by the server 106. It will be appreciated that the user device 132 may also include the heart rate algorithm 110c, and may receive physiological data from the wearable device 104 in order to perform some/all of the functionality described herein.

Although two (2) electronic devices 104, 132 and one (1) server 106 are depicted in FIG. 1A, it should be appreciated that greater or fewer amounts are envisioned. For example, there may be multiple servers, each one associated with a different entity. Moreover, it is to be appreciated that a computer program product in accordance with an aspect may include a computer usable storage medium (e.g., standard random access memory (RAM), an optical disc, a universal serial bus (USB) drive, or the like) having computer-readable program code embodied therein, wherein the computer-readable program code may be adapted to be executed by the processors 112, 122 (e.g., working in connection with the respective operating systems 110a, 120a) to facilitate the functions as described herein. In this regard, the program code may be implemented in any desired language, and may be implemented as machine code, assembly code, byte code, interpretable source code or the like (e.g., via Golang, Python, Scala, C, C++, Java, Actionscript, Objective-C, Javascript, CSS, XML). In some aspects, the computer program product may be part of a cloud network of resources.

FIG. 1B illustrates an example parameter fit 140 corresponding to physiological parameters obtained from a wearable device of a user (e.g., wearable device 104), in accordance with various aspects disclosed herein. Generally, the example parameter fit 140 illustrates example physiological data 142 collected from a user, and example parameter fits 144 (e.g., physiological parameters) corresponding to the data 142.

The example physiological data 142 may generally represent heart rate data and steps data of a user. Of course, the example physiological data 142 may include data as collected by any sensor included as part of a wearable (e.g., the set of sensors 108), but the heart rate data and steps data are very commonly measured by a wide range of consumer-grade wearables (e.g., smartwatches, such as wearable device 104). In particular, the heart rate data is a physiologic signal that, in conjunction with the techniques of the present disclosure, may be used to, for example, track disease progression, detect imminent symptom onset, and distinguish between positive and negative cases of common infections (e.g., COVID-19). However, changes in the heart rate data occurring specifically from disease may become obscured by other factors that may affect heart rate on a daily timescale or shorter timescale, such as circadian variation, activity, stress, hormones, caffeine and meals, sleep, and posture.

To overcome the effects of these factors, the heart rate algorithm (e.g., heart rate algorithm 110c) may generate the example parameter fits 144 based on the example physiological data 142. Generally, the heart rate algorithm may fit example parameter fits 144 to the example physiological data 142 to account for variations in the heart rate data due to each of the other factors previously described and/or any other suitable physiological factors that may influence heart rate. For example, the example parameter fits 144 may be broadly grouped into parameterization categories that account for variations in a wearable user's (e.g., user 102) heart rate data due to (i) the user's circadian rhythm in heart rate (CRHR), (ii) changes in the user's physical activity levels, and (iii) acute changes to the user's heart rate as a result of other miscellaneous factors (e.g., eating, light changes, stress, etc.). Moreover, in some aspects, data acquired by a wearable device (e.g., wearable device 104) during sleep is discarded to remove the masking effects of sleep on the collected physiological data. Alternatively, in certain aspects, the wearable device may adjust the data acquired during sleep to account for the effects of sleep on the acquired data and accordingly incorporate the data acquired during sleep into the example parameter fits 144.

More specifically, the example parameter fits 144 may represent a daily circadian profile fit to the example physiological data 142 (e.g., continuous heart rate data, steps data) along with a linear coefficient that corresponds to how much a wearable user's (e.g., user 102) heart rate increases per step (e.g., physical activity). Of course, it is to be appreciated that the example parameter fits 144 may represent circadian profile fits over any suitable timescale, such as every hour, every eight hours, every sixteen hours, every day, every two days, every week, etc.

The example parameter fits 144 may also include a noise parameter that reflects the strength of acute changes in a user's heart rate that occur on timescales shorter than 24 hours to account for miscellaneous factors beyond circadian and physical activity-related variation. The noise parameter may be split into a correlated noise parameter and an uncorrelated noise parameter. Correlated noise could be effects on a user's heart rate resulting from stress, meals, caffeine, light, posture, hormones, etc., while uncorrelated noise may impact the user's heart rate as a result of device noise or other external effects.

The heart rate algorithm may generate each of the parameters included in the example parameter fits 144 to fit the following heart rate function:

HR=a−b·cos(x(Time−c))+d Steps+ε (1)

where HR is the user's heart rate at a particular time, Time is a particular time or time interval during a day, and Steps is the number of steps taken by the user at the particular time or time interval. Further, in equation (1), the parameter a may correspond to a user's average heart rate, the parameter b may correspond to an amplitude of a user's circadian variation, x may be a constant value related to the circadian variation phase, the parameter c may correspond to a phase of a user's circadian variation, the parameter d may correspond to the effect of a single step on a user's heart rate (also referenced herein as “heart rate per step” (HRpS)), and the parameter ε may correspond to the total noise. In some aspects, the parameter E may be sub-divided into additional parameters k and a. In these aspects, the parameter k may correspond to the autocorrelated noise parameter, and the parameter a may correspond to an independent (uncorrelated) noise parameter.

Of course, it is to be appreciated that the heart rate algorithm 110c may fit any number of parameters that may correspond to any suitable physiological signals, and the model represented in equation (1) may be of any suitable order. Moreover, the parameter fits (e.g., example parameter fits 144) may be grouped into hierarchical models and/or any other suitable grouping. It is also to be appreciated that the wearable device and/or server may apply the heart rate algorithm 110c to collected physiological data at any suitable frequency, such as every minute, every five minutes, every hour, every day, and/or any other suitable frequency or combinations thereof.

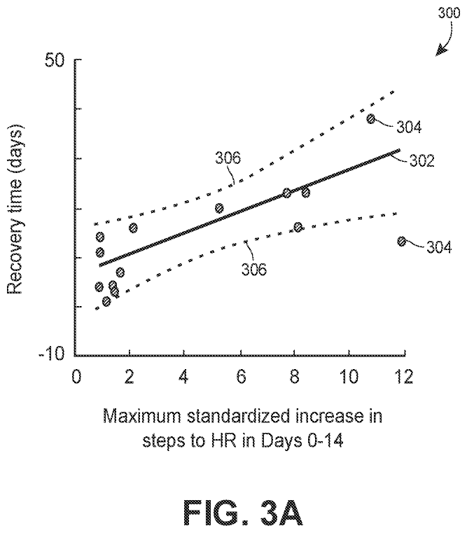

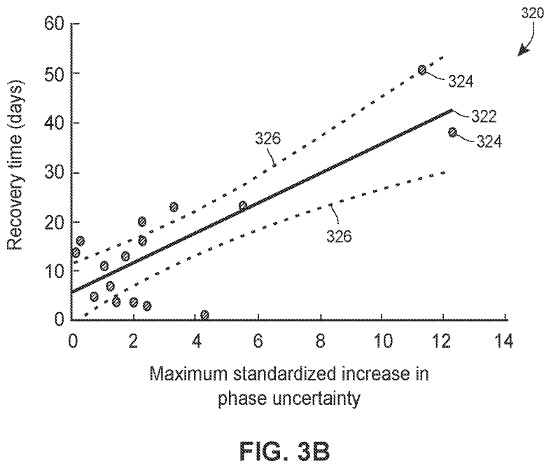

Further, it should be understood that the heart rate algorithm 110c may calculate confidence intervals and/or uncertainty values corresponding to each parameter, such as illustrated in FIGS. 3A (e.g., 306) and 3B (e.g., 326). The confidence intervals and/or uncertainty values may generally indicate how accurately the calculations performed by the heart rate algorithm 110c approximate each individual parameter and/or the calculated parameters as an aggregate collection at a particular time. Higher confidence intervals and/or lower uncertainty values may influence the analysis/determinations performed by one or more processors of the present techniques. More generally, these confidence intervals and/or uncertainty values may be used at various points in the methods and systems described herein. For example, the confidence intervals and/or uncertainty values calculated by the heart rate algorithm 110c may be used as part of analyzing the one or more physiological parameters to output a period classification and/or determining whether or not the period classification is indicative of an infection. To illustrate, if the parameters calculated by the heart rate algorithm 110c typically indicate an infection, but the accompanying confidence intervals are low and/or the uncertainty values are high, then the resulting period classification may not clearly indicate an infection because the heart rate algorithm 110c cannot conclusively determine that the parameters exceed and/or otherwise fail to satisfy an infection threshold.

FIG. 1C illustrates an example physiological data collection and parameterization timeline 150 corresponding to the example system of FIG. 1A, and in accordance with various aspects disclosed herein. Generally, as illustrated in FIG. 1C, the heart rate algorithm 110c may be applied (e.g., by the wearable device 104 and/or the server 106) to extract parameter fits (e.g., the example parameter fits 144) on a daily basis during each of a calibration period 152, a patient baseline period 154, and an analysis period 156, during which, the patient may develop/experience symptoms as a result of a disease/infection. Symptom onset may generally refer to a period during which a patient develops known symptoms associated with a particular disease/infection, and/or may correspond to a positive test associated with the particular disease/infection. For example, the patient may develop symptoms of a particular disease at day 0, which may thereafter be regarded as the date of symptom onset 156a.

During the calibration period 152, the wearable device may acquire and utilize physiological data in order to train or otherwise calibrate the heart rate algorithm to generate physiological parameters and/or period classifications. For example, the wearable device may detect, store, and apply physiological data (e.g., heart rate data and steps data) to the heart rate algorithm during the calibration period 152 to train the heart rate algorithm to recognize physiological data that should be removed from a data set of a particular day (e.g., physiological data representative of a sleep period of a user). As another example, the wearable device may utilize physiological data collected during the calibration period 152 to calibrate the heart rate algorithm to properly evaluate variability in the user's physiological data over several days/weeks so that the heart rate algorithm may properly recognize when physiological data is genuinely indicative of a disease/infection for a particular user. The calibration period 152 may last (e.g., the wearable device may train the heart rate algorithm) for several weeks/months to properly train the heart rate algorithm to generate parameter fits and/or period classifications corresponding to the physiological data.

Regardless, it is to be understood that the calibration period 152 may include training or otherwise calibrating the heart rate algorithm to generate physiological parameters and/or period classifications based on any suitable data. In some aspects, the calibration period 152 may include calibrating the heart rate algorithm using data corresponding to one or more respective physiological systems of the user that may each produce a physiological impact on the heart rate of the user. For example, the one or more respective physiological systems may include one or more of: (i) circadian timekeeping, (ii) respiratory function related to increased heart rate from activity of the user, (iii) sleep, (iv) hormones, (v) meal consumption, (vi) caffeine intake, or (vii) posture.

During the patient baseline period 154, the wearable device may acquire and utilize physiological data in order to develop/generate a baseline set of physiological parameters corresponding to the user wearing the wearable device using the heart rate algorithm. The wearable device may apply the heart rate algorithm to compare the baseline set of physiological parameters to physiological data/parameters detected and/or generated during a symptomatic period of a user's infection. In this manner, the heart rate algorithm may accurately detect when a user has contracted a disease/infection by determining that the physiological data (and the corresponding physiological parameters generated thereby) deviates significantly (e.g., more than a threshold amount) from the baseline set of physiological parameters. For example, the wearable device may detect, store, and apply physiological data (e.g. heart rate data and steps data) for several weeks to develop the patient-specific baseline set of physiological parameters using the heart rate algorithm.

During the analysis period 156, the wearable device may acquire and utilize physiological data to determine whether or not a user has contracted a disease/infection by applying the heart rate algorithm to the physiological data. Generally, the wearable device may apply the heart rate algorithm to the physiological data acquired during the analysis period 156 to generate and compare physiological parameters to the corresponding parameters included as part of the patient-specific baseline set of physiological parameters. As previously mentioned, if the heart rate algorithm determines that the physiological parameters generated based on the physiological data collected during the analysis period 156 deviate more than a threshold amount from the physiological parameters included in the patient-specific baseline set of physiological parameters, the heart rate algorithm may output a period classification indicating that the user of the wearable device has contracted a disease/infection. The wearable device may detect, store, and apply physiological data (e.g. heart rate data and steps data) for a few days/weeks to determine whether or not a user has contacted a disease/infection using the heart rate algorithm. Of course, for each of the periods 152, 154, 156, the wearable device may detect and/or store physiological data for any suitable length of time, such as minutes, hours, days, weeks, months, years, etc.

As an example, the calibration period 152 may begin 50 days prior to symptom onset and may extend through 34 days prior to symptom onset. In this example, the patient baseline period 154 may begin 34 days prior to symptom onset and may extend through 7 days prior to symptom onset. Further in this example, the analysis period 156 may begin 7 days prior to symptom onset and may extend through 14 days after symptom onset. However, it is to be understood that the calibration period 152, the patient baseline period 154, and the analysis period 156 may be any suitable periods of any suitable length, and the heart rate algorithm 110c may calculate parameter fits in accordance with the techniques disclosed herein during any suitable number of periods.

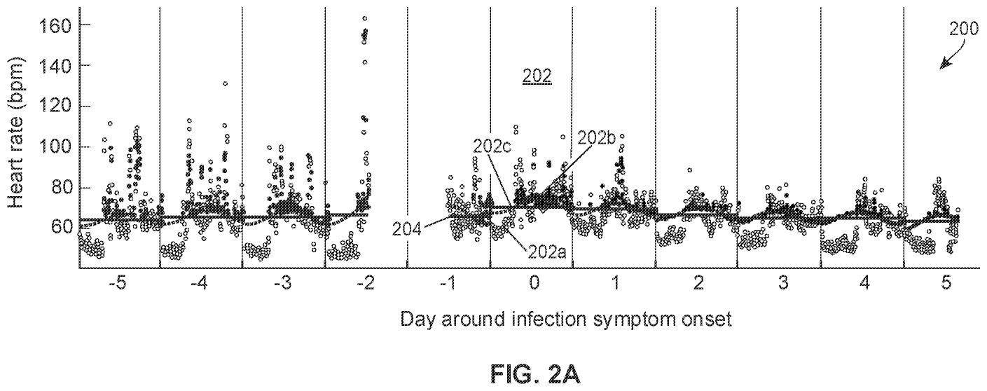

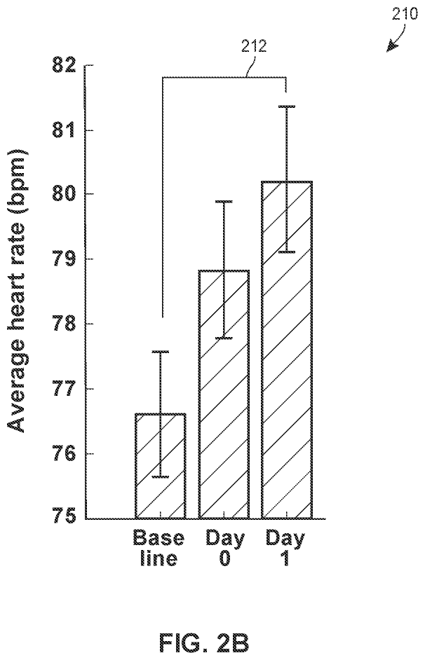

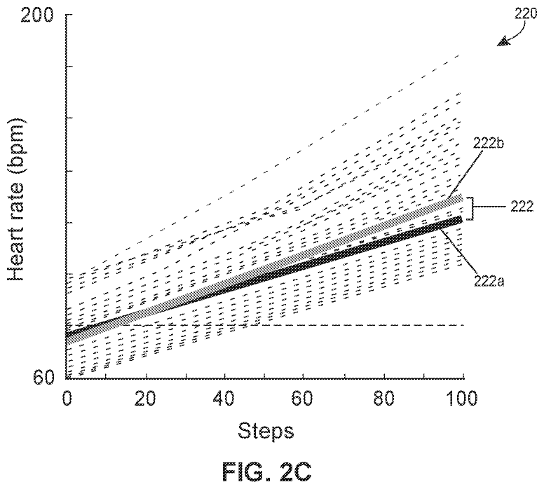

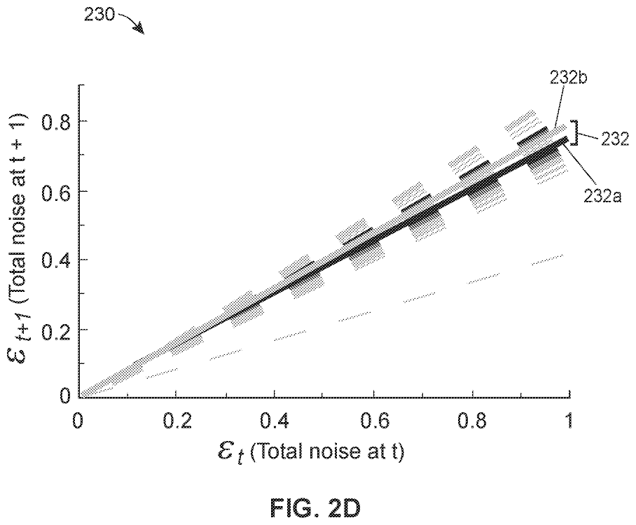

FIG. 2A is a graph 200 illustrating an example infection detection utilizing the wearable device of the example system of FIG. 1A, and in accordance with various aspects disclosed herein. Generally, the graph 200 illustrates three distinct heart rate data sets resulting from physiological data collected by the wearable device. Each of these heart rate data sets are represented across multiple days that are defined in the graph 200 relative to a date of symptom onset 202. A first heart rate data set 202a represents raw heart rate data collected by a wearable device during each of the days illustrated on the graph 200. A second heart rate data set 202b represents estimated heart rate values calculated using equation (1) using the raw heart rate data from the first heart rate data set 202a. A third heart rate data set 202c represents a daily average heart rate calculated by, for example, the wearable device as a result of removing various effects such as circadian timekeeping, sleep, and activity from the second heart rate data set 202b.

In order to detect whether or not a wearable device user has contracted a disease/infection, the wearable device may utilize the heart rate algorithm to analyze, generate, and/or compare the heart rate data sets 202a, 202b, 202c across multiple days. Broadly speaking, the daily average heart rate (as represented by the third heart rate data set 202c) tends to increase on either the day of symptom onset (e.g., day 0) or the day after (e.g., day 1). For example, the heart rate data sets 202a, 202b, 202c generally elevate on day 0 relative to the surrounding days illustrated in FIG. 2A, in response to the onset of symptoms of a disease/infection.

To highlight this effect, FIG. 2B provides a graph 210 illustrating example changes in a user's heart rate, as measured by a wearable device, that occur during and after the onset of an infection. Generally, the graph 210 includes three average heart rate values 212 and their associated error bars. The three average heart rate values 212 include an average heart rate value for a user's baseline (e.g., as calculated during a patient baseline period 154), an average heart rate value for the user on the day of symptom onset (e.g., day 0), and an average heart rate value for the user on the day after the day of symptom onset (e.g., day 1). Each of these values may be a mean heart rate value calculated by the wearable device by averaging heart rate values detected throughout each respective day, and/or the wearable device may calculate the average heart rate values using any suitable statistical averaging technique. In any event, as illustrated in FIG. 2B, the average heart rate value during the baseline period may be approximately 76.6 beats per minute (bpm), the average heart rate value on day 0 may be approximately 78.8 bpm, and the average heart rate value on day 1 may be approximately 80.2 bpm. Thus, the wearable device may, by applying the heart rate algorithm to calculate the physiological parameters and thereby calculate the average heart rate values 212, determine that the mean individual daily average heart rate for the user is significantly higher on day 1 relative to that of the baseline period, for example, with a p-value of 0.02 resulting from a two-tailed t-test. As a result, the wearable device may also generate a period classification indicating that the user is symptomatic and may have contracted a disease/infection, as discussed further herein.

As another example, FIG. 2C is a graph 220 illustrating example changes in a user's heart rate due to steps (e.g., HRpS), as measured by a wearable device, that occur during and after the onset of an infection, in accordance with various aspects disclosed herein. Generally, the HRpS parameter may increase after symptom onset. However, the average HRpS value for a user in the analysis period (e.g., analysis period 156) may not always be significantly higher than that in the baseline period (e.g., patient baseline period 154). Nevertheless, during the two weeks after symptom onset (e.g., day 0), most wearable users experience an increase in the HRpS parameter relative to their respective baseline, and many wearable users exhibited a significant increase relative to their respective baseline.

Generally, the graph 220 illustrates two linear fits 222 representing the HRpS parameter (e.g., d), as calculated by the heart rate algorithm, over two distinct periods: the user's baseline period (fit 222a), and the user's analysis period after symptom onset (fit 222b). In particular, as part of the period classification analysis the wearable device and/or server applying the heart rate algorithm may fit a linear regression to the HRpS parameter data for every day in the baseline period and every day after symptom onset (represented by the dotted lines in FIG. 2C). From these linear fits, the wearable device and/or server may compute a mean linear relationship for heart rate and steps in the baseline period (e.g., fit 222a) and the period after symptom onset (e.g., fit 222b). The heart rate algorithm may then analyze, for example, the slope of the fits 222a, 222b to determine whether or not the fit 222b is indicative of a wearable user having contracted a disease/infection. If the slope of fit 222b exceeds the slope of fit 222a by more than a threshold slope value, then the heart rate algorithm may determine that the HRpS parameter for the wearable user over the period represented by the fit 222b indicates that the user is symptomatic.

In yet another example, FIG. 2D is a graph 230 illustrating example changes in the autocorrelated noise of a user's physiological data, as included in measurements of the wearable device, that occur during and after the onset of an infection, in accordance with various aspects disclosed herein. Generally, the autocorrelated noise parameter represents the noise at a time t+1 that is carried over from a time t. Thus, the autocorrelated noise parameter may reflect changes in a user's heart rate due to factors other than steps and inherent circadian variation (e.g., hormone release due to stress, prolonged standing, meals, light, caffeine, etc.). At the time of symptom onset (e.g., day 0), the autocorrelated noise parameter may increase in users, similar to the increase in the average heart rate values on days 0 and 1 illustrated in FIG. 2B. For example, the wearable device may apply the heart rate algorithm to the autocorrelated noise parameter (e.g., parameter k included as part of parameter E in equation (1)) to determine a period classification. The wearable device applying the heart rate algorithm may detect these changes in the autocorrelated noise parameter, and may output a period classification indicating that the user is symptomatic and may have contracted a disease/infection, as discussed further herein.

Namely, the graph 230 includes two linear models 232 fit by the wearable device and/or server to residuals at time t+1 relative to t on days in the baseline period (e.g., fit 232a) and on the date of symptom onset (e.g., fit 232b). The wearable device and/or server may fix the intercept at zero as the uncorrelated noise may be normally distributed about 0. In any event, the slope of the fit 232b representative of the date of symptom onset (e.g., day 0) is significantly higher than the slope of the fit 232a representative of the user's baseline. Thus, the autocorrelated noise generally increases on the date of symptom onset relative to the baseline period, such that the wearable device applying the heart rate algorithm may determine period classifications based, in part, on the changes in slope of fits (e.g., 232a, 232b) corresponding to the autocorrelation noise parameter.

It is to be appreciated that the wearable device, by applying the heart rate algorithm, may evaluate/analyze any of the physiological parameters calculated as a result of the collected physiological data to determine a period classification. Further, it is to be appreciated that the heart rate algorithm may output a period classification indicating that the user is non-symptomatic (e.g., healthy) despite the fact that an individual physiological parameter may indicate that the user is symptomatic. For example, assume that the uncorrelated noise parameter of a user exhibits a general decrease during a first period relative to the user's baseline period value for the uncorrelated noise parameter, such that the uncorrelated noise parameter evaluated independently may indicate that the user is symptomatic for a disease/infection. However, further assume that none of the other physiological parameters indicate that the user is symptomatic during the first period. In this example, the heart rate algorithm may output a period classification indicating that the user is non-symptomatic over the first period despite the general decrease of the uncorrelated noise parameter during the first period.

In addition to determining period classifications using the physiological data collected by the wearable device, the heart rate algorithm may also calculate/determine predicted recovery times to help users track the progress of and/or to approximate the severity of a contracted disease/infection using the physiological data. Generally, these recovery time predictions are based on similar analyses as the period classifications, in that the heart rate algorithm may analyze physiological data collected after symptom onset to generate physiological parameters and thereby track how a user's physiological data is progressing throughout the course of a contracted disease/infection. Based on this analysis, the heart rate algorithm may provide frequent and/or updated recovery time predictions using updated/recent physiological parameters generated from updated/recent physiological data.

As an example, FIG. 3A is a graph 300 illustrating an example recovery time estimation, generated by a wearable device and/or server, based on a user's maximum increase in heart rate per step (HRpS) during an infection. As illustrated in FIG. 3A, a user's maximum increase in HRpS during a period of infection (illustrated here as 2 weeks for simplicity) bears a significant linear relationship to the recovery time for that user. Specifically, the linear fit 302 may represent the approximate relationship between these two values, as determined by performing a linear regression using the data points 304 within certain error tolerances 306.

Using this relationship, the wearable device may collect heart rate data and steps data throughout a user's period of infection, and the heart rate algorithm may determine and analyze a user's maximum increase in HRpS at various points throughout the infection period to generate predicted recovery times. As the user's maximum increase in HRpS changes during their period of infection, the heart rate algorithm may predict different recovery times. For example, assume that a user's maximum increase in HRpS during their first three days of experiencing disease/infection symptoms is 2 HRpS compared to the user's baseline. In this example, the heart rate algorithm may generate a recovery time prediction of approximately 10 days based on the linear fit 302. However, further assume that six days after symptom onset, the user experiences an increase in HRpS of 8 HRpS compared to the user's baseline. In this case, the heart rate algorithm may revise the recovery time prediction to 20 days in accordance with the linear fit 302.

As another example, FIG. 3B is a graph 320 illustrating an example recovery time estimation, generated by a wearable device and/or server, based on a user's maximum phase uncertainty increase during an infection. As illustrated in FIG. 3B, a user's maximum phase uncertainty increase (e.g., corresponding to the circadian variation phase parameter) during a period of infection bears a significant linear relationship to the recovery time for that user. Specifically, the linear fit 322 may represent the approximate relationship between these two values, as determined by performing a linear regression using the data points 324 within certain error tolerances 326.

Using this relationship, the wearable device may collect heart rate data and steps data throughout a user's period of infection, and the heart rate algorithm may determine and analyze a user's maximum phase uncertainty increase at various points throughout the infection period to generate predicted recovery times. As the user's maximum phase uncertainty increase changes during their period of infection, the heart rate algorithm may predict different recovery times. For example, assume that a user's maximum phase uncertainty increase during their first two days of experiencing disease/infection symptoms is 4 compared to the user's baseline. In this example, the heart rate algorithm may generate a recovery time prediction of approximately 15 days based on the linear fit 322. However, further assume that seven days after symptom onset, the user experiences an increase in phase uncertainty of 10 compared to the user's baseline. In this case, the heart rate algorithm may revise the recovery time prediction to 35 days in accordance with the linear fit 322.

Of course, it should be appreciated that each example described herein utilizing a linear regression or fitting technique may be accomplished using any suitable statistical fitting technique. Moreover, while the examples provided above in reference to FIGS. 2A-3B describe particular instances of the heart rate algorithm calculating and/or determining values using specific physiological data and/or physiological parameters, it is to be understood that the heart rate algorithm may utilize any of the physiological data and/or parameters described herein as part of any suitable calculation or determination. Each of the example graphs illustrated in reference to FIGS. 2A-3B may additionally or alternatively be generated and/or rendered based on calculations/determinations performed by a server (e.g., server 106) or a user device (e.g., wearable device 104, user device 132).

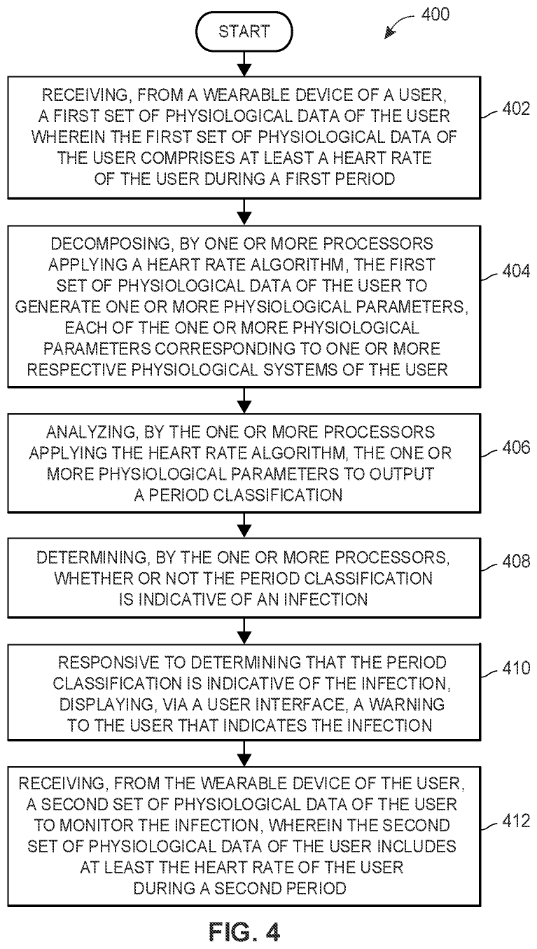

FIG. 4 illustrates an example method 400 for enhancing infection detection and monitoring through decomposed physiological data, in accordance with various aspects disclosed herein. For ease of discussion, many of the various actions included in the method 400 are described herein as performed by or with the use of a wearable device (e.g., wearable device 104). However, it is to be appreciated that the various actions included in the method 400 may be performed by, for example, a wearable device (e.g., wearable device 104), a user device (e.g., user device 132), a server (e.g., server 106), and/or other suitable processors or combinations thereof.

In any event, the method 400 may include receiving, from a wearable device of a user, a first set of physiological data of the user (block 402). The first set of physiological data of the user may comprise at least a heart rate of the user during a first period. In certain aspects, the first set of physiological data of the user may comprise the heart rate of the user, an active step number of the user, and a temperature of the user.

The physiological data may be binned or transformed to target specific timescales and/or to average out faster timescales, variability, or wearable device noise to target specific physiological systems. In some aspects, the first set of physiological data may comprise a plurality of first physiological data points. In these aspects, each respective first physiological data point may be binned into one of a plurality of bins, and each respective bin may represent a five minute interval of the first period. For example, the wearable device may constantly and/or intermittently collect data via a heart rate sensor, tri-axial accelerometer, and/or any other suitable sensor, and this collected data may be binned/aggregated into distinct intervals defined by time periods of interest. A first set of physiological data collected by the wearable device during a first five minute interval may be binned into a first bin representative of the first five minute interval. Similarly, a second set of physiological data collected by the wearable device during a second five minute interval that is subsequent to the first five minute interval may be binned into a second bin representative of the second five minute interval. Further, in these aspects, the wearable device, by applying the heart rate algorithm, may calculate physiological parameters for each five minute interval. Moreover, each five minute interval may be included as part of a larger period of interest (e.g., individual days, months, etc.), over which the wearable device, by applying the heart rate algorithm, may calculate physiological parameters representative of the larger period of interest based on the physiological data in each bin included within the larger period of interest.

The method 400 further includes decomposing, by one or more processors applying a heart rate algorithm, the first set of physiological data of the user to generate one or more physiological parameters (block 404). Each of the one or more physiological parameters may correspond to one or more respective physiological systems of the user. Generally, the wearable device may decompose the physiological data of a user into multiple components representing different physiological systems by analyzing the physiological data in regards to a specific physiological system of the user. The signals from separate physiological systems may interact in an additive, multiplicative, and/or correlative (e.g., shaping the correlation between residuals at different times) manner. Thus, the wearable device may identify a specific physiological system, and utilize data corresponding to other systems as part of the residuals. For example, the wearable device may calculate/predict the effect of steps on a user's circadian clock, and the wearable device may use this relationship to refine the heart rate's circadian signal.

In some aspects, the one or more physiological parameters include at least (i) an average heart rate of the user (e.g., parameter a) and one or more of: (ii) a circadian variation amplitude (e.g., parameter b), (iii) a circadian variation phase (e.g., parameter c), (iv) an activity parameter (e.g., parameter d), (v) an autocorrelated noise parameter (e.g., parameter k), or (vi) an uncorrelated noise parameter (e.g., parameter a).

In certain aspects, the one or more respective physiological systems of the user may each produce a physiological impact on the heart rate of the user. For example, the one or more respective physiological systems may include one or more of: (i) circadian timekeeping, (ii) respiratory function related to increased heart rate from activity of the user, (iii) sleep, (iv) hormones, (v) meal consumption, (vi) caffeine intake, or (vii) posture.

In some aspects, the wearable device may additionally determine, by applying the heart rate algorithm, a set of sleep heart rate data from the first set of physiological data of the user. The set of sleep heart rate data may be captured by the wearable device of the user during a sleep period of the first period (e.g., during nighttime hours when the user is asleep). In these aspects, the wearable device may remove the set of sleep heart rate data from the first set of physiological data of the user.

In certain aspects, the heart rate algorithm may be an artificial intelligence (AI) based algorithm configured to execute one or more ML models. In these aspects, the wearable device and/or a server may train the heart rate algorithm with a set of healthy period training data and a set of symptomatic period training data to output respective physiological parameters and respective period classifications. Generally, the set of healthy period training data may correspond to physiological data obtained from a plurality of individuals prior to experiencing symptoms of a respective disease/infection, and the set of symptomatic period training data may correspond to physiological data obtained from a plurality of individuals while each individual is experiencing symptoms of the respective disease/infection. The heart rate algorithm may also be trained with the set of healthy period training data and the set of symptomatic period training data to output recovery time predictions and/or disease/infection severity predictions, as discussed herein. Further in these aspects, the heart rate algorithm may be a linear support-vector machine (SVM) model.

The method 400 may further include analyzing, by the one or more processors applying the heart rate algorithm, the one or more physiological parameters to output a period classification (block 406). As previously mentioned, the period classification may generally indicate whether or not a user is symptomatic with a particular disease/infection. In certain aspects, the wearable device may determine the period classification, in part, by comparing the one or more physiological parameters to a user-specific baseline using the heart rate algorithm.

For example, the wearable device may determine, by applying the heart rate algorithm, whether or not any deviations of the one or more physiological parameters from the user-specific baseline exceed an infection threshold. Responsive to determining that at least one deviation of a respective physiological parameter from the user-specific baseline exceeds the infection threshold, the wearable device may generate a warning to the user that indicates the infection. Further in these aspects, the user-specific baseline may include one or more respective baseline physiological parameters, and each of the one or more respective baseline physiological parameters may correspond to a respective physiological parameter of the one or more physiological parameters.

The method 400 may also include determining whether or not the period classification is indicative of an infection (block 408). Responsive to determining that the period classification is indicative of an infection, the wearable device may display a warning to the user that indicates the infection in a user interface (block 410). The user interface may be an integrated component of the wearable device and/or the wearable device may transmit the warning to a separate device (e.g., user device 132) for display on a user interface of the separate device. In certain aspects, responsive to determining that the period classification is indicative of the infection, the wearable device may determine a recovery time of the user from the infection based on at least one respective physiological parameter of the one or more physiological parameters.

The method 400 may further include receiving, from the wearable device of the user, a second set of physiological data of the user to monitor the infection (block 412). Generally, the second set of physiological data of the user may include at least the heart rate data of the user during a second period, and the second period may occur any suitable length of time following the first period. The wearable device of the user may receive the second set of physiological data, generate new physiological parameters based on the second set of physiological data, and may compare the newly generated physiological parameters to the physiological parameters generated based on the first set of physiological data.

In some aspects, the one or more physiological parameters comprises a first set of one or more physiological parameters. Further in these aspects, the wearable device may decompose the second set of physiological data of the user to generate a second set of one or more physiological parameters by applying the heart rate algorithm. Each of the second set of one or more physiological parameters may correspond to the one or more respective physiological systems of the user. As a result, the wearable device may update the recovery time of the user based on the second set of one or more physiological parameters. Of course, in aspects where the wearable device generates a disease/infection severity prediction, and/or any other output, the wearable device may additionally or alternatively update those generated outputs.

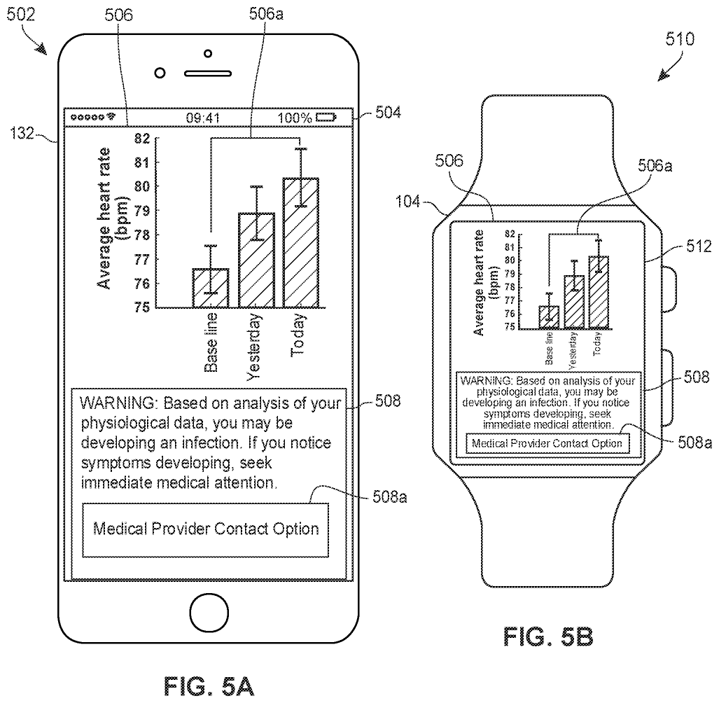

When displaying warnings, predictions, and/or other outputs, the wearable device may provide displays similar to those presented in FIGS. 5A and 5B. FIG. 5A illustrates an example user interface 502 as rendered on a display screen of a user device (e.g., user device 132). For example, as shown in the example of FIG. 5A, the user interface 502 may be implemented or rendered via an application (app) executing on the user device 132. The user interface 502 may be implemented or rendered via a native app executing on the user device 132.

In the example of FIG. 5A, the user device 132 is a user device as described for FIG. 1A, e.g., where 132 is illustrated as an APPLE iPhone that implements the APPLE iOS operating system and that has display screen 504. The user device 132 may execute one or more native applications (apps) on its operating system, and such native apps may be implemented or coded (e.g., as computing instructions) in a computing language (e.g., SWIFT) executable by the user device 132 operating system (e.g., APPLE iOS) by the processor of user device 132. Additionally, or alternatively, the user interface 502 may be implemented or rendered via a web interface, such as via a web browser application, e.g., Safari and/or Google Chrome app(s), or other such web browser or the like.

As shown in the example of FIG. 5A, the user interface 502 comprises a graphical representation 506 (e.g., similar to graph 210) of a user's heart rate during three distinct periods 506a. The graphical representation 506 may comprise physiological parameters of the user (or graphical representations thereof), as described herein. In the example of FIG. 5A, the graphical representation 506 is annotated with one or more graphics or textual rendering(s) (e.g., axis labels) corresponding to the physiological parameters identifiable within the graphical representation 506. Additionally, graphics, colors, scores, patterns, and/or other renderings may be annotated or overlaid on top of the graphical representation 506 to highlight the feature(s) of interest within the representation 506 by the heart rate algorithm. In the example of FIG. 5A, the physiological parameters represented by the three distinct periods 506a indicate a trending increase in the user's heart rate, and may indicate other physiological symptoms, as described herein.

The user interface 502 may also include or render a user-specific message 508 based on the data presented in the graphical representation 506. In the embodiment of FIG. 5A, the user-specific message 508 comprises a warning to the user designed to provide a brief description of a period classification output by the heart rate algorithm. As shown in the example of FIG. 5A, the message 508 indicates to a user that based on the analysis of the user's physiological data, the user may be developing an infection. Accordingly, the message 508 suggests that if the user notice symptoms developing, that the user should seek immediate medical attention.

The user-specific message 508 may also include or render a medical provider contact option 508a, which may be a selectable UI button configured to provide the user with contact options for various medical providers in response to receiving input (e.g., selection) from the user. For example, a user may select the medical provider contact option 508a, and the application may retrieve and display phone numbers, websites, instant chat, telemedicine links, email addresses, and/or any other suitable contact option that a user may utilize to contact a medical provider.

Similarly, FIG. 5B illustrates another example user interface 510 as rendered on a display screen of a wearable device of the user (e.g., wearable device 104). The example user interface 510 includes the graphical representation 506 (e.g., similar to graph 210) of a user's heart rate during three distinct periods 506a, the user-specific message 508, and the medical provider contact option 508a. Of course, the wearable device 104 may display each of the representation 506, the message 508, and the contact option 508a in a different format and/or rendering layout than the user device 132 due to dimensional differences of the respective display screens 504, 512.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connects the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of the example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but also deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but also deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

The foregoing description is given for clearness of understanding; and no unnecessary limitations should be understood therefrom, as modifications within the scope of the invention may be apparent to those having ordinary skill in the art.

What is claimed:

1. A method for enhancing infection detection and monitoring through decomposed physiological data, the method comprising:
receiving, from a wearable device of a user, a first set of physiological data of the user, wherein the first set of physiological data of the user comprises at least heart rate data of the user during a first period;
decomposing, by one or more processors applying a heart rate algorithm, the first set of physiological data of the user to generate one or more physiological parameters, each of the one or more physiological parameters corresponding to one or more respective physiological systems of the user;
analyzing, by the one or more processors applying the heart rate algorithm, the one or more physiological parameters to output a period classification;
determining, by the one or more processors, whether or not the period classification is indicative of an infection;
responsive to determining that the period classification is indicative of the infection, displaying, in a user interface, a warning to the user that indicates the infection; and
receiving, from the wearable device of the user, a second set of physiological data of the user to monitor the infection, wherein the second set of physiological data of the user includes at least the heart rate data of the user during a second period.

2. The method of claim 1, wherein the one or more physiological parameters comprises at least (i) an average heart rate of the user and one or more of: (ii) a circadian variation amplitude, (iii) a circadian variation phase, (iv) an activity parameter, (v) an autocorrelated noise parameter, or (vi) an uncorrelated noise parameter.

3. The method of claim 1, wherein the first set of physiological data of the user comprises the heart rate of the user, an active step number of the user, and a temperature of the user.

4. The method of claim 1, further comprising:
responsive to determining that the period classification is indicative of the infection, determining, by the one or more processors, a recovery time of the user from the infection based on at least one respective physiological parameter of the one or more physiological parameters.

5. The method of claim 4, wherein the one or more physiological parameters comprises a first set of one or more physiological parameters, the method further comprising:
decomposing, by one or more processors applying the heart rate algorithm, the second set of physiological data of the user to generate a second set of one or more physiological parameters, wherein each of the second set of one or more physiological parameters corresponds to the one or more respective physiological systems of the user; and
updating, by the one or more processors, the recovery time of the user based on the second set of one or more physiological parameters.

6. The method of claim 1, wherein decomposing the first set of physiological data of the user further comprises:
determining, by the one or more processors applying the heart rate algorithm, a set of sleep heart rate data from the first set of physiological data of the user that was captured by the wearable device of the user during a sleep period of the first period; and
removing, by the one or more processors, the set of sleep heart rate data from the first set of physiological data of the user.

7. The method of claim 1, wherein the first set of physiological data comprises a plurality of first physiological data points, each respective first physiological data point is binned into one of a plurality of bins, and each respective bin represents a five minute interval of the first period.

8. The method of claim 1, wherein analyzing the one or more physiological parameters further comprises:
comparing, by the one or more processors applying the heart rate algorithm, the one or more physiological parameters to a user-specific baseline;
determining, by the one or more processors applying the heart rate algorithm, whether or not any deviations of the one or more physiological parameters from the user-specific baseline exceed an infection threshold; and
responsive to determining that at least one deviation of a respective physiological parameter from the user-specific baseline exceeds the infection threshold, generating, by the one or more processors, the warning to the user that indicates the infection.

9. The method of claim 8, wherein the user-specific baseline includes one or more respective baseline physiological parameters, each of the one or more respective baseline physiological parameters corresponding to a respective physiological parameter of the one or more physiological parameters.

10. The method of claim 1, wherein the one or more respective physiological systems of the user each produce a physiological impact on the heart rate of the user, and the one or more respective physiological systems comprise one or more of: (i) circadian timekeeping, (ii) respiratory function related to increased heart rate from activity of the user, (iii) sleep, (iv) hormones, (v) meal consumption, (vi) caffeine intake, or (vii) posture.

11. The method of claim 1, wherein the heart rate algorithm is an artificial intelligence (AI) based algorithm, and the method further comprises:
training, by the one or more processors, the heart rate algorithm with a set of healthy period training data and a set of symptomatic period training data to output respective physiological parameters and respective period classifications.

12. The method of claim 11, wherein the heart rate algorithm is a linear support-vector machine (SVM) model.

13. A system for enhancing infection detection and monitoring through decomposed physiological data, the system comprising:
a user interface;
a wearable device of a user configured to capture a first set of physiological data of the user, wherein the first set of physiological data of the user comprises at least heart rate data of the user during a first period;
a memory storing a set of computer-readable instructions comprising at least a heart rate algorithm; and
a processor interfacing with the user interface, the wearable device, and the memory, and configured to execute the set of computer-readable instructions to cause the processor to:
receive, from the wearable device of the user, the first set of physiological data of the user,
decompose, by applying the heart rate algorithm, the first set of physiological data of the user to generate one or more physiological parameters, each of the one or more physiological parameters corresponding to one or more respective physiological systems of the user,
analyze, by applying the heart rate algorithm, the one or more physiological parameters to output a period classification,
determine whether or not the period classification is indicative of an infection,
responsive to determining that the period classification is indicative of the infection, display, in the user interface, a warning to the user that indicates the infection, and
receive, from the wearable device of the user, a second set of physiological data of the user to monitor the infection, wherein the second set of physiological data of the user includes at least the heart rate data of the user during a second period.

14. The system of claim 13, wherein the one or more physiological parameters comprises at least (i) an average heart rate of the user and one or more of: (ii) a circadian variation amplitude, (iii) a circadian variation phase, (iv) an activity parameter, (v) an autocorrelated noise parameter, or (vi) an uncorrelated noise parameter.

15. The system of claim 13, wherein the first set of physiological data of the user comprises the heart rate of the user, an active step number of the user, and a temperature of the user.

16. The system of claim 13, wherein the set of computer-readable instructions further cause the processor to:
responsive to determining that the period classification is indicative of the infection, determine a recovery time of the user from the infection based on at least one respective physiological parameter of the one or more physiological parameters.

17. The system of claim 13, wherein the set of computer-readable instructions further cause the processor to:
determine, by applying the heart rate algorithm, a set of sleep heart rate data from the first set of physiological data of the user that was captured by the wearable device of the user during a sleep period of the first period; and
remove the set of sleep heart rate data from the first set of physiological data of the user.

18. The system of claim 13, wherein the first set of physiological data comprises a plurality of first physiological data points, each respective first physiological data point is binned into one of a plurality of bins, and each respective bin represents a five minute interval of the first period.

19. The system of claim 13, wherein the set of computer-readable instructions further cause the processor to:
compare the one or more physiological parameters to a user-specific baseline, wherein the user-specific baseline includes one or more respective baseline physiological parameters, each of the one or more respective baseline physiological parameters corresponding to a respective physiological parameter of the one or more physiological parameters;
determine whether or not any deviations of the one or more physiological parameters from the user-specific baseline exceed an infection threshold; and
responsive to determining that at least one deviation of a respective physiological parameter from the user-specific baseline exceeds the infection threshold, generate the warning to the user that indicates the infection.

20. A non-transitory computer-readable storage medium having stored thereon a set of instructions, executable by at least one processor, for enhancing infection detection and monitoring through decomposed physiological data, the instructions comprising:
instructions for accessing a first set of physiological data of a user, wherein the first set of physiological data of the user comprises at least heart rate data of the user during a first period;
instructions for decomposing, by applying a heart rate algorithm, the first set of physiological data of the user to generate one or more physiological parameters, each of the one or more physiological parameters corresponding to one or more respective physiological systems of the user;
instructions for analyzing, by applying the heart rate algorithm, the one or more physiological parameters to output a period classification;
instructions for determining whether or not the period classification is indicative of an infection;
responsive to determining that the period classification is indicative of the infection, instructions for displaying, in a user interface, a warning to the user that indicates the infection; and
instructions for accessing a second set of physiological data of the user to monitor the infection, wherein the second set of physiological data of the user includes at least the heart rate data of the user during a second period.
